(19) United States
(12) Patent Application Publication
(10) Pub. No.: US 2022/0110896 A1
(43) Pub. Date: Apr. 14, 2022

(54) COMPOSITIONS AND METHODS FOR PROVIDING SUSTAINED NATURAL SLEEP

(71) Applicant: FarmaKeio Nutraceuticals, LLC, Richardson, TX (US)

(72) Inventors: Dustin DeNeui, Richardson, TX (US); Justin Graves, Richardson, TX (US)

(73) Assignee: FarmaKeio Nutraceuticals, LLC, Richardson, TX (US)

(21) Appl. No.: 17/557,019

(22) Filed: Dec. 20, 2021

Related U.S. Application Data
(63) Continuation-in-part of application No. 16/372,723, filed on Apr. 2, 2019.
(60) Provisional application No. 62/652,190, filed on Apr. 3, 2018.

(51) Int. Cl. A61K 31/197; A61K 36/575; A61K 36/84; A61K 31/4045

(52) U.S. Cl. CPC A61K 31/197; A61K 31/4045; A61K 36/84; A61K 36/575

(57) ABSTRACT

Disclosed are compositions which provide sustained natural sleep. Further disclosed are methods for providing natural sleep without the side effect of drowsiness.

### CLAIM TO PRIORITY

This Application is a Continuation-In-Part of application Ser. No. 16/372,723, filed Apr. 2, 2019, which claims priority to U.S. Provisional Application Ser. No. 62/652,190, filed Apr. 3, 2018, the entirety of which is included herein by reference.

### FIELD

Disclosed are compositions which provide sustained natural sleep. Further disclosed are methods for providing natural sleep without the side effect of drowsiness.

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Unless otherwise indicated, the definitions and embodiments described in this and other sections are intended to be applicable to all embodiments and aspects of the present application herein described for which they are suitable as would be understood by a person skilled in the art.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a compound” should be understood to present certain aspects with one compound or two or more additional compounds.

The term “and/or” as used herein means that the listed items are present, or used, individually or in combination. In effect, this term means that “at least one of” or “one or more” of the listed items is used or present.

The term “naturally occurring” means “an unprocessed chemical occurring in nature or natural environment, being a substance that is extracted by manual, mechanical, or gravitational means; or dissolution in water; or flotation; or a process of heating for the sole purpose of removing uncombined water; without a chemical change in the substance.”

The term “subject” as used herein includes all members of the animal kingdom including mammals, and suitably refers to humans, companion animals (e.g. dogs, cats, rodents, rabbits etc.) and livestock (e.g. cattle, sheep, pigs, goats, equines such as horses, mules and donkeys etc.).

The term “pharmaceutically acceptable” means compatible with the treatment of subjects such as humans, companion animals and livestock.

The term “administered” as used herein means administration of an effective amount of the disclosed sleep aides to subject.

Disclosed herein are sleep aide compositions which provide natural sleep to the user without the side effect of drowsiness. The disclosed compositions comprise:
A) from about 70% to about 90% by weight of active ingredients; and
B) from about 10% to about 30% by weight of carriers, excipients, adjunct ingredients, and formulation aides.

“Natural sleep” is defined herein as sleep that would be obtained without the aid of a pharmaceutically active, for example, a prescription sleep aid. Sustained natural sleep includes no waking during the desired sleep period and include waking when the predetermined sleep period ends, for example, when awakened by an alarm. The sustained natural sleep includes wakening without drowsiness or a latent feeling of being “drugged.”

In addition, natural sleep includes the ability to be awakened during the desired sleep period and not having a sense of disorientation, drowsiness or lack of energy. The disclosed compositions when self-administered allow the subject to predetermine the length of the sleep cycle.

The following is a listing of the naturally occurring active ingredients which comprise the disclosed sleep aides.

### Aminophenylbutyric Acid

The disclosed compositions comprise from about 25% to about 38% by weight of aminophenylbutyric acid hydrochloride. In one embodiment the disclosed compositions comprise from about 27% to about 32% by weight of aminophenylbutyric acid hydrochloride. In another embodiment the disclosed compositions comprise from about 27% to about 30% by weight of aminophenylbutyric acid hydrochloride.

The disclosed compositions can comprise any amount by weight of aminophenylbutyric hydrochloride from about 25% to about 38% by weight, for example, 25%, 26%, 27%, 28%, 29%, 30%, 31%, 32%, 33%, 34%, 35%, 36%, 37% or 38%. In addition, the compositions can comprise any fractional percentage from about 25% to about 38%, for example, 29.12%, 25.24% and the like.

In one aspect, the disclosed compositions comprise from about 120 mg to about 200 mg by weight of aminophenylbutyric acid hydrochloride (phenibut) or another pharmaceutically acceptable salt of aminophenylbutyric acid. In one embodiment the disclosed compositions comprise about 135 mg to about 200 mg by weight of aminophenylbutyric acid hydrochloride. In another embodiment the disclosed compositions comprise from about 120 mg to about 180 mg by weight of aminophenylbutyric acid hydrochloride.

In a further embodiment the disclosed compositions comprise from about 140 mg to about 200 mg by weight of aminophenylbutyric acid hydrochloride. In a yet further embodiment the disclosed compositions comprise from about 120 mg to about 160 mg by weight of aminophenylbutyric acid hydrochloride. In a yet another embodiment the disclosed compositions comprise from about 145 mg to about 200 mg by weight of aminophenylbutyric acid hydrochloride. In a still further embodiment the disclosed compositions comprise from about 145 mg to about 155 mg by weight of aminophenylbutyric acid hydrochloride. In a non-limiting example, the compositions comprise about 150 mg of aminophenylbutyric acid hydrochloride.

The disclosed compositions can comprise any amount by weight of aminophenylbutyric acid hydrochloride is from about 120 mg to about 200 mg, for example, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199, or 200 mg.

### Magnolia Bark Extract

The Magnolia bark extract which comprises the disclosed sleep aides, contains about 2% by weight of honokiol. Honokiol, (2-(4-hydroxy-3-prop-2-enylphenyl)-4-prop-2-enylphenol), is a lignin isolated from the bark, seed cones, and leaves of trees belonging to the genus Magnolia having the formula:

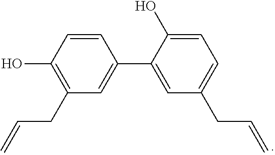 [SMILES: C=CCc1ccc(O)c(-c2ccc(O)c(CC=C)c2)c1]

The disclosed compositions comprise from about 15% to about 28% by weight of magnolia bark extract. In one embodiment the disclosed compositions comprise from about 17% to about 22% by weight of magnolia bark extract. In another embodiment the disclosed compositions comprise from about 17% to about 20% by weight of magnolia bark extract.

The disclosed compositions can comprise any amount by weight of magnolia bark extract from about 15% to about 28% by weight, for example, 15%, 16%, 17%, 18%, 19%, 20%, 21%, 22%, 23%, 24%, 25%, 26%, 27% or 28%. In addition, the compositions can comprise any fractional percentage from about 15% to about 25%, for example, 19.41%, 21.24% and the like.

In one aspect, the disclosed compositions comprise from about 60 mg to about 140 mg by weight of magnolia bark extract form Magnolia officinalis. In one embodiment the disclosed compositions comprise about 70 mg to about 140 mg by weight of magnolia bark extract. In another embodiment the disclosed compositions comprise from about 60 mg to about 130 mg by weight of magnolia bark extract. In a further embodiment the disclosed compositions comprise from about 75 mg to about 140 mg by weight of magnolia bark extract. In a yet further embodiment the disclosed compositions comprise from about 75 mg to about 120 mg by weight of magnolia bark extract. In a yet another embodiment the disclosed compositions comprise from about 90 mg to about 120 mg by weight of magnolia bark extract. In a still further embodiment the disclosed compositions comprise from about 90 mg to about 110 mg by weight of magnolia bark extract. In a non-limiting example, the compositions comprise about 100 mg of magnolia bark extract.

The disclosed compositions can comprise any amount by weight of magnolia bark extract from about 60 mg to about 140 mg, for example, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139, or 140 mg.

### Valerian Root

Valerian root is a naturally occurring material which has sedative properties. It is obtained from the dried roots of various valerian species, for example, Valeiana officianalis. The disclosed compositions comprise from about 15% to about 25% by weight of valerian root. In one embodiment the disclosed compositions comprise from about 17% to about 22% by weight of valerian root. In another embodiment the disclosed compositions comprise from about 17% to about 20% by weight of valerian root.

The disclosed compositions can comprise any amount by weight of valerian root from about 15% to about 25% by weight, for example, 15%, 16%, 17%, 18%, 19%, 20%, 21%, 22%, 23%, 24% or 25%. In addition, the compositions can comprise any fractional percentage from about 15% to about 25%, for example, 0.097%, 0.18% and the like.

In one aspect, the disclosed compositions comprise from about 60 mg to about 140 mg by weight of valerian root. In one embodiment the disclosed compositions comprise about 70 mg to about 140 mg by weight of valerian root. In another embodiment the disclosed compositions comprise from about 60 mg to about 130 mg by weight of valerian root. In a further embodiment the disclosed compositions comprise from about 75 mg to about 140 mg by weight of valerian root. In a yet further embodiment the disclosed compositions comprise from about 75 mg to about 120 mg by weight of valerian root. In a yet another embodiment the disclosed compositions comprise from about 90 mg to about 120 mg by weight of valerian root. In a still further embodiment the disclosed compositions comprise from about 90 mg to about 110 mg by weight of valerian root. In a non-limiting example, the compositions comprise about 100 mg of valerian root.

The disclosed compositions can comprise any amount by weight of valerian root from about 60 mg to about 140 mg, for example, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139, or 140 mg.

### 5-Hydroxytryptophan

The disclosed compositions comprise from about 5% to about 15% by weight of 5-hydroxytryptophan. In one embodiment the disclosed compositions comprise from about 7% to about 12% by weight of 5-hydroxytryptophan. In another embodiment the disclosed compositions comprise from about 8% to about 11% by weight of 5-hydroxytryptophan.

The disclosed compositions can comprise any amount by weight of 5-hydroxytryptophan from about 5% to about 15% by weight, for example, 5%, 6%, 7%, 18%, 9%, 10%, 11%, 12%, 13%, 14% or 15%. In addition, the compositions can comprise any fractional percentage from about 5% to about 15%, for example, 9.7%, 10.02% and the like.

In one aspect, the disclosed compositions comprise from about 20 mg to about 80 mg by weight of 5-hydroxytryptophan. In one embodiment the disclosed compositions comprise about 30 mg to about 80 mg by weight of 5-hydroxytryptophan. In another embodiment the disclosed compositions comprise from about 20 mg to about 70 mg by weight of 5-hydroxytryptophan. In a further embodiment the disclosed compositions comprise from about 35 mg to about 80 mg by weight of 5-hydroxytryptophan. In a yet further embodiment the disclosed compositions comprise from about 35 mg to about 70 mg by weight of 5-hydroxytryptophan. In a yet another embodiment the disclosed compositions comprise from about 40 mg to about 60 mg by weight of 5-hydroxytryptophan. In a still further embodiment the disclosed compositions comprise from about 45 mg to about 55 mg by weight of 5-hydroxytryptophan. In a non-limiting example, the compositions comprise about 50 mg of 5-hydroxytryptophan.

The disclosed compositions can comprise any amount by weight of 5-hydroxytryptophan from about 20 mg to about 80 mg, for example, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, or 80 mg.

### Melatonin

The disclosed compositions can comprise from about 0.05% to about 0.25% by weight of melatonin. In one embodiment the compositions comprise from about 0.075% to about 0.15% by weight of melatonin. In another embodiment the compositions comprise from about 0.09% to about 0.12% by weight of melatonin.

The disclosed compositions can comprise any amount by weight of melatonin from about 0.05% to about 0.25% by weight, for example, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24% or 0.25%. In addition, the compositions can comprise any fractional percentage from about 5% to about 15%, for example, 9.7%, 10.02% and the like.

In one aspect, the disclosed compositions comprise from about 0.02 mg to about 2 mg by weight of melatonin (N-acetyl-5-methoxytryptamine). In one embodiment the disclosed compositions comprise about 0.1 mg to about 2 mg by weight of melatonin. In another embodiment the disclosed compositions comprise from about 0.1 mg to about 1.5 mg by weight of melatonin. In a further embodiment the disclosed compositions comprise from about 0.25 mg to about 2 mg by weight of melatonin. In a yet further embodiment the disclosed compositions comprise from about 0.25 mg to about 1 mg by weight of melatonin. In a yet another embodiment the disclosed compositions comprise from about 0.35 mg to about 0.65 mg by weight of melatonin. In a still further embodiment the disclosed compositions comprise from about 0.45 mg to about 0.55 mg by weight of melatonin. In a non-limiting example, the compositions comprise about 0.5 mg of melatonin.

The disclosed compositions can comprise any amount by weight of melatonin from about 0.02 mg to about 2 mg, for example, 0.1 mg, 0.11 mg, 0.12 mg, 0.13 mg, 0.14 mg, 0.15 mg, 0.15 mg, 0.17 mg, 0.18 mg, 0.19 mg, 0.2 mg, 0.21 mg, 0.22 mg, 0.23 mg, 0.24 mg, 0.25 mg, 0.25 mg, 0.27 mg, 0.28 mg, 0.29 mg, 0.3 mg, 0.31 mg, 0.32 mg, 0.33 mg, 0.34 mg, 0.35 mg, 0.35 mg, 0.37 mg, 0.38 mg, 0.39 mg, 0.4 mg, 0.41 mg, 0.42 mg, 0.43 mg, 0.44 mg, 0.45 mg, 0.45 mg, 0.47 mg, 0.48 mg, 0.49 mg, 0.5 mg, 0.51 mg, 0.52 mg, 0.53 mg, 0.54 mg, 0.55 mg, 0.55 mg, 0.57 mg, 0.58 mg, 0.59 mg, 0.6 mg, 0.61 mg, 0.62 mg, 0.63 mg, 0.64 mg, 0.65 mg, 0.65 mg, 0.67 mg, 0.68 mg, 0.69 mg, 0.7 mg, 0.71 mg, 0.72 mg, 0.73 mg, 0.74 mg, 0.75 mg, 0.75 mg, 0.77 mg, 0.78 mg, 0.79 mg, 0.8 mg, 0.81 mg, 0.82 mg, 0.83 mg, 0.84 mg, 0.85 mg, 0.85 mg, 0.87 mg, 0.88 mg, 0.89 mg, 0.9 mg, 0.91 mg, 0.92 mg, 0.93 mg, 0.94 mg, 0.95 mg, 0.95 mg, 0.97 mg, 0.98 mg, 0.99 mg, or 1 mg.

### Compositions

In one embodiment the disclosed compositions comprise:
A) from about 75% to about 85% by weight of an active ingredient component, comprising:
   a) from about 32% to about 38% by weight of aminophenylbutyric acid hydrochloride;
   b) from about 24.5% to about 28% by weight of magnolia bark;
   c) from about 24.5% to about 28% by weight of valerian root;
   d) from about 12% to about 14% by weight of 5-HTP; and
   e) from about 0.1 to about 1% by weight of melatonin; and
B) from about 15% to about 25% by weight carriers, excipients, and formulation aides.

A non-limiting example of this embodiment, comprises:
A) an active ingredient component, comprising:
   a) about 140 mg of aminophenylbutyric acid hydrochloride;
   b) about 110 mg of magnolia bark;
   c) about 100 mg of valerian root;
   d) about 50 mg of 5-HTP; and
   e) about 0.5 mg of melatonin; and
B) about 98.5 mg carriers, excipients, and formulation aides.

A further non-limiting example of this embodiment, comprises:
A) an active ingredient component, comprising:
   a) about 130 mg of aminophenylbutyric acid hydrochloride;
   b) about 110 mg of magnolia bark;
   c) about 110 mg of valerian root;
   d) about 50 mg of 5-HTP; and
   e) about 0.5 mg of melatonin; and
B) about 98.5 mg carriers, excipients, and formulation aides.

Another non-limiting example of this embodiment, comprises:
A) an active ingredient component, comprising:
   a) about 140 mg of aminophenylbutyric acid hydrochloride;
   b) about 100 mg of magnolia bark;
   c) about 99.5 mg of valerian root;
   d) about 50 mg of 5-HTP; and
   e) about 1 mg of melatonin; and
B) about 98.5 mg carriers, excipients, and formulation aides.

A non-limiting example of this embodiment, comprises:
A) an active ingredient component, comprising:
   a) about 150 mg of aminophenylbutyric acid hydrochloride;
   b) about 99.5 mg of magnolia bark;
   c) about 100 mg of valerian root;
   d) about 50 mg of 5-HTP; and
   e) about 0.5 mg of melatonin; and
B) about 98.5 mg carriers, excipients, and formulation aides.

In another embodiment the disclosed compositions comprise:
A) from about 75% to about 80% by weight of an active ingredient component, comprising:
   a) from about 32% to about 38% by weight of aminophenylbutyric acid hydrochloride;
   b) from about 24.5% to about 28% by weight of magnolia bark;
   c) from about 24.5% to about 28% by weight of valerian root;
   d) from about 12% to about 13% by weight of 5-HTP; and
   e) from about 0.1 to about 0.5% by weight of melatonin; and
B) from about 20% to about 25% by weight carriers, excipients, and formulation aides.

A non-limiting example of this embodiment, comprises:
A) an active ingredient component, comprising:
   a) about 140 mg of aminophenylbutyric acid hydrochloride;
   b) about 110 mg of magnolia bark;
   c) about 100 mg of valerian root;
   d) about 50 mg of 5-HTP; and
   e) about 0.5 mg of melatonin; and
B) about 114.5 mg carriers, excipients, and formulation aides.

A further non-limiting example of this embodiment, comprises:
A) an active ingredient component, comprising:
   a) about 130 mg of aminophenylbutyric acid hydrochloride;
   b) about 110 mg of magnolia bark;
   c) about 110 mg of valerian root;
   d) about 50 mg of 5-HTP; and
   e) about 0.5 mg of melatonin; and
B) about 114.5 mg carriers, excipients, and formulation aides.

Another non-limiting example of this embodiment, comprises:
A) an active ingredient component, comprising:
   a) about 150 mg of aminophenylbutyric acid hydrochloride;
   b) about 100 mg of magnolia bark;
   c) about 99.5 mg of valerian root;
   d) about 50 mg of 5-HTP; and
   e) about 1 mg of melatonin; and
B) about 114.5 mg carriers, excipients, and formulation aides.

A still further non-limiting example of this embodiment, comprises:
A) an active ingredient component, comprising:
   a) about 150 mg of aminophenylbutyric acid hydrochloride;
   b) about 100 mg of magnolia bark;
   c) about 100 mg of valerian root;
   d) about 50 mg of 5-HTP; and
   e) about 0.5 mg of melatonin; and
B) about 114.5 mg carriers, excipients, and formulation aides.

In a further embodiment the disclosed compositions comprise:
A) from about 70% to about 80% by weight of an active ingredient component, comprising:
   a) from about 34.5% to about 39% by weight of aminophenylbutyric acid hydrochloride;
   b) from about 24.5% to about 28% by weight of magnolia bark;
   c) from about 24.5% to about 25.5% by weight of valerian root;
   d) from about 12% to about 13% by weight of 5-HTP; and
   e) from about 0.1 to about 0.5% by weight of melatonin; and
B) from about 20% to about 25% by weight carriers, excipients, and formulation aides.

A non-limiting example of this embodiment, comprises:
A) an active ingredient component, comprising:
   a) about 150 mg of aminophenylbutyric acid hydrochloride;
   b) about 100 mg of magnolia bark;
   c) about 100 mg of valerian root;
   d) about 50 mg of 5-HTP; and
   e) about 0.5 mg of melatonin; and
B) about 129.5 mg carriers, excipients, and formulation aides.

A further non-limiting example of this embodiment, comprises:
A) an active ingredient component, comprising:
   a) about 160 mg of aminophenylbutyric acid hydrochloride;
   b) about 110 mg of magnolia bark;
   c) about 110 mg of valerian root;
   d) about 50 mg of 5-HTP; and
   e) about 0.5 mg of melatonin; and
B) about 129.5 mg carriers, excipients, and formulation aides.

Another non-limiting example of this embodiment, comprises:
A) an active ingredient component, comprising:
   a) about 140 mg of aminophenylbutyric acid hydrochloride;
   b) about 100 mg of magnolia bark;
   c) about 99.5 mg of valerian root;
   d) about 50 mg of 5-HTP; and
   e) about 1 mg of melatonin; and
B) about 129.5 mg carriers, excipients, and formulation aides.

A non-limiting example of this embodiment, comprises:
A) an active ingredient component, comprising:
   a) about 150 mg of aminophenylbutyric acid hydrochloride;
   b) about 99.5 mg of magnolia bark;
   c) about 100 mg of valerian root;
   d) about 50 mg of 5-HTP; and
   e) about 0.5 mg of melatonin; and
B) about 129.5 mg carriers, excipients, and formulation aides.

The following Tables provide non-limiting examples of the disclosed compositions. All amounts are in milligrams (mg).

TABLE I

| Ingredients | Example No. | | | | |
|---|---|---|---|---|---|
| Amounts in mg | 1 | 2 | 3 | 4 | 5 |
| Phenibut* | 140 | 130 | 150 | 150 | 140 |
| Magnolia bark | 110 | 110 | 100 | 99.5 | 110 |
| extract | | | | | |
| Valeria root | 100 | 110 | 99.5 | 100 | 110 |
| 5-HTP | 50 | 50 | 50 | 50 | 40 |
| Melatonin | 0.5 | 0.5 | 1 | 1 | 1 |
| Excipients** | 98.5 | 98.5 | 98.5 | 98.5 | 98.5 |

*As the hydrochloride salt.
**microcrystalline cellulose, magnesium stearate, silicon dioxide.

TABLE II

| Ingredients | Example No. | | | | |
|---|---|---|---|---|---|
| Amounts in mg | 6 | 7 | 8 | 9 | 10 |
| Phenibut* | 130 | 130 | 130 | 130 | 140 |
| Magnolia bark | 100 | 100 | 100 | 99.5 | 100 |
| extract | | | | | |
| Valeria root | 100 | 110 | 99.5 | 100 | 100 |
| 5-HTP | 50 | 55 | 50 | 50 | 50 |
| Melatonin | 2 | 2 | 2.5 | 2.5 | 2.5 |
| Excipients** | 98.5 | 98.5 | 98.5 | 98.5 | 98.5 |

*As the hydrochloride salt.
**microcrystalline cellulose, magnesium stearate, silicon dioxide.

TABLE III

| Ingredients | Example No. | | | | |
|---|---|---|---|---|---|
| Amounts in mg | 11 | 12 | 13 | 14 | 15 |
| Phenibut* | 130 | 130 | 130 | 130 | 140 |
| Magnolia bark | 100 | 100 | 100 | 99.5 | 100 |
| extract | | | | | |
| Valeria root | 100 | 110 | 99.5 | 100 | 100 |
| 5-HTP | 50 | 55 | 50 | 50 | 50 |
| Melatonin | 3 | 3 | 3 | 3 | 3 |
| Excipients** | 98.5 | 98.5 | 98.5 | 98.5 | 98.5 |

*As the hydrochloride salt.
**microcrystalline cellulose, magnesium stearate, silicon dioxide.

TABLE IV

| Ingredients | Example No. | | | | |
|---|---|---|---|---|---|
| Amounts in mg | 16 | 17 | 18 | 19 | 20 |
| Phenibut* | 140 | 130 | 150 | 150 | 140 |
| Magnolia bark | 110 | 110 | 100 | 100 | 110 |
| extract | | | | | |
| Valeria root | 100 | 110 | 99.5 | 100 | 110 |
| 5-HTP | 50 | 50 | 50 | 50 | 40 |
| Melatonin | 0.5 | 0.5 | 1 | 0.5 | 1 |
| Excipients** | 114.5 | 114.5 | 114.5 | 114.5 | 114.5 |

*As the hydrochloride salt.
**microcrystalline cellulose, magnesium stearate, silicon dioxide.

TABLE V

| Ingredients | Example No. | | | | |
|---|---|---|---|---|---|
| Amounts in mg | 21 | 22 | 23 | 24 | 25 |
| Phenibut* | 150 | 160 | 140 | 140 | 140 |
| Magnolia bark | 100 | 110 | 100 | 90 | 110 |
| extract | | | | | |
| Valeria root | 100 | 110 | 90 | 100 | 110 |
| 5-HTP | 50 | 50 | 50 | 50 | 40 |
| Melatonin | 0.5 | 0.5 | 1 | 0.5 | 1 |
| Excipients** | 129.5 | 129.5 | 129.5 | 129.5 | 129.5 |

*As the hydrochloride salt.
**microcrystalline cellulose, magnesium stearate, silicon dioxide.

TABLE VI

| Ingredients | Example No. | | | | |
|---|---|---|---|---|---|
| Amounts in mg | 26 | 27 | 28 | 29 | 30 |
| Phenibut* | 150 | 160 | 140 | 140 | 140 |
| Magnolia bark | 100 | 110 | 100 | 90 | 110 |
| extract | | | | | |
| Valeria root | 100 | 110 | 90 | 100 | 110 |
| 5-HTP | 50 | 50 | 50 | 50 | 40 |
| Melatonin | 1 | 1 | 2 | 2 | 2 |
| Excipients** | 129.5 | 129.5 | 129.5 | 129.5 | 129.5 |

*As the hydrochloride salt.
**microcrystalline cellulose, magnesium stearate, silicon dioxide.

A further embodiment the disclosed compositions comprise:
A) from about 75% to about 85% by weight of an active ingredient component, comprising:
   a) from about 32% to about 38% by weight of γ-aminobutyric acid hydrochloride;
   b) from about 24.5% to about 28% by weight of magnolia bark;
   c) from about 24.5% to about 28% by weight of valerian root;
   d) from about 12% to about 14% by weight of 5-HTP; and
   e) from about 0.1 to about 1% by weight of melatonin; and
B) from about 15% to about 25% by weight carriers, excipients, and formulation aides.

A non-limiting example of this embodiment, comprises:
A) an active ingredient component, comprising:
   a) about 100 mg of γ-aminobutyric acid hydrochloride;
   b) about 110 mg of magnolia bark;
   c) about 100 mg of valerian root;
   d) about 50 mg of 5-HTP; and
   e) about 0.5 mg of melatonin; and
B) about 98.5 mg carriers, excipients, and formulation aides.

A further non-limiting example of this embodiment, comprises:
A) an active ingredient component, comprising:
   a) about 130 mg of γ-aminobutyric acid hydrochloride;
   b) about 110 mg of magnolia bark;
   c) about 110 mg of valerian root;
   d) about 50 mg of 5-HTP; and
   e) about 0.5 mg of melatonin; and
B) about 98.5 mg carriers, excipients, and formulation aides.

Another non-limiting example of this embodiment, comprises:
A) an active ingredient component, comprising:
   a) about 140 mg of γ-aminobutyric acid hydrochloride;
   b) about 100 mg of magnolia bark;
   c) about 99.5 mg of valerian root;
   d) about 50 mg of 5-HTP; and
   e) about 1 mg of melatonin; and
B) about 98.5 mg carriers, excipients, and formulation aides.

A non-limiting example of this embodiment, comprises:
A) an active ingredient component, comprising:
   a) about 150 mg of γ-aminobutyric acid hydrochloride;
   b) about 99.5 mg of magnolia bark;
   c) about 100 mg of valerian root;
   d) about 50 mg of 5-HTP; and
   e) about 0.5 mg of melatonin; and
B) about 98.5 mg carriers, excipients, and formulation aides.

In another embodiment the disclosed compositions comprise:
A) from about 75% to about 80% by weight of an active ingredient component, comprising:
   a) from about 32% to about 38% by weight of γ-aminobutyric acid hydrochloride;
   b) from about 24.5% to about 28% by weight of magnolia bark;
   c) from about 24.5% to about 28% by weight of valerian root;
   d) from about 12% to about 13% by weight of 5-HTP; and
   e) from about 0.1 to about 0.5% by weight of melatonin; and
B) from about 20% to about 25% by weight carriers, excipients, and formulation aides.

A non-limiting example of this embodiment, comprises:
A) an active ingredient component, comprising:
   a) about 100 mg of γ-aminobutyric acid hydrochloride;
   b) about 110 mg of magnolia bark;
   c) about 100 mg of valerian root;
   d) about 50 mg of 5-HTP; and
   e) about 0.5 mg of melatonin; and
B) about 114.5 mg carriers, excipients, and formulation aides.

A further non-limiting example of this embodiment, comprises:
A) an active ingredient component, comprising:
   a) about 130 mg of γ-aminobutyric acid hydrochloride;
   b) about 110 mg of magnolia bark;
   c) about 110 mg of valerian root;
   d) about 50 mg of 5-HTP; and
   e) about 0.5 mg of melatonin; and
B) about 114.5 mg carriers, excipients, and formulation aides.

Another non-limiting example of this embodiment, comprises:
A) an active ingredient component, comprising:
   a) about 150 mg of γ-aminobutyric acid hydrochloride;
   b) about 100 mg of magnolia bark;
   c) about 99.5 mg of valerian root;
   d) about 50 mg of 5-HTP; and
   e) about 1 mg of melatonin; and
B) about 114.5 mg carriers, excipients, and formulation aides.

A still further non-limiting example of this embodiment, comprises:
A) an active ingredient component, comprising:
   a) about 150 mg of γ-aminobutyric acid hydrochloride;
   b) about 100 mg of magnolia bark;
   c) about 100 mg of valerian root;
   d) about 50 mg of 5-HTP; and
   e) about 0.5 mg of melatonin; and
B) about 114.5 mg carriers, excipients, and formulation aides.

In a further embodiment the disclosed compositions comprise:
A) from about 70% to about 80% by weight of an active ingredient component, comprising:
   a) from about 34.5% to about 39% by weight of γ-aminobutyric acid hydrochloride;
   b) from about 24.5% to about 28% by weight of magnolia bark;
   c) from about 24.5% to about 25.5% by weight of valerian root;
   d) from about 12% to about 13% by weight of 5-HTP; and
   e) from about 0.1 to about 0.5% by weight of melatonin; and
B) from about 20% to about 25% by weight carriers, excipients, and formulation aides.

A non-limiting example of this embodiment, comprises:
A) an active ingredient component, comprising:
   a) about 100 mg of γ-aminobutyric acid hydrochloride;
   b) about 100 mg of magnolia bark;
   c) about 100 mg of valerian root;
   d) about 50 mg of 5-HTP; and
   e) about 0.5 mg of melatonin; and
B) about 129.5 mg carriers, excipients, and formulation aides.

A further non-limiting example of this embodiment, comprises:
A) an active ingredient component, comprising:
   a) about 160 mg of γ-aminobutyric acid hydrochloride;
   b) about 110 mg of magnolia bark;
   c) about 110 mg of valerian root;
   d) about 50 mg of 5-HTP; and
   e) about 0.5 mg of melatonin; and
B) about 129.5 mg carriers, excipients, and formulation aides.

Another non-limiting example of this embodiment, comprises:
A) an active ingredient component, comprising:
   a) about 140 mg of γ-aminobutyric acid hydrochloride;
   b) about 100 mg of magnolia bark;
   c) about 99.5 mg of valerian root;
   d) about 50 mg of 5-HTP; and
   e) about 1 mg of melatonin; and
B) about 129.5 mg carriers, excipients, and formulation aides.

A non-limiting example of this embodiment, comprises:
A) an active ingredient component, comprising:
   a) about 150 mg of γ-aminobutyric acid hydrochloride;
   b) about 99.5 mg of magnolia bark;
   c) about 100 mg of valerian root;
   d) about 50 mg of 5-HTP; and
   e) about 0.5 mg of melatonin; and
B) about 129.5 mg carriers, excipients, and formulation aides.

TABLE VII

| | |
|---|---|
| Ingredients | Example No. |

| Amounts in mg | 31 | 32 | 33 | 34 | 35 |
|---|---|---|---|---|---|
| γ-aminobutyric acid | 140 | 100 | 100 | 100 | 140 |
| Magnolia bark | 110 | 110 | 100 | 99.5 | 110 |
| extract | | | | | |
| Valeria root | 100 | 110 | 99.5 | 100 | 110 |
| 5-HTP | 50 | 50 | 50 | 50 | 40 |
| Melatonin | 0.5 | 0.5 | 1 | 1 | 1 |
| Excipients** | 98.5 | 98.5 | 98.5 | 98.5 | 98.5 |

**microcrystalline cellulose, magnesium stearate, silicon dioxide.

TABLE VIII

| | |
|---|---|
| Ingredients | Example No. |

| Amounts in mg | 36 | 37 | 38 | 39 | 40 |
|---|---|---|---|---|---|
| γ-aminobutyric acid | 130 | 130 | 100 | 130 | 140 |
| Magnolia bark | 100 | 100 | 100 | 99.5 | 100 |
| extract | | | | | |
| Valeria root | 100 | 110 | 99.5 | 100 | 100 |
| 5-HTP | 50 | 55 | 50 | 50 | 50 |
| Melatonin | 2 | 2 | 2.5 | 2.5 | 2.5 |
| Excipients** | 98.5 | 98.5 | 98.5 | 98.5 | 98.5 |

**microcrystalline cellulose, magnesium stearate, silicon dioxide.

TABLE IX

| | |
|---|---|
| Ingredients | Example No. |

| Amounts in mg | 41 | 42 | 43 | 44 | 45 |
|---|---|---|---|---|---|
| γ-aminobutyric acid | 100 | 120 | 99 | 100 | 100 |
| Magnolia bark | 100 | 100 | 100 | 99.5 | 100 |
| extract | | | | | |
| Valeria root | 100 | 110 | 99.5 | 100 | 100 |
| 5-HTP | 50 | 55 | 50 | 50 | 50 |
| Melatonin | 3 | 3 | 3 | 3 | 3 |
| Excipients** | 98.5 | 98.5 | 98.5 | 98.5 | 98.5 |

**microcrystalline cellulose, magnesium stearate, silicon dioxide.

TABLE X

| | |
|---|---|
| Ingredients | Example No. |

| Amounts in mg | 46 | 47 | 48 | 49 | 50 |
|---|---|---|---|---|---|
| γ-aminobutyric acid | 110 | 130 | 110 | 100 | 100 |
| Magnolia bark | 110 | 110 | 100 | 100 | 110 |
| extract | | | | | |
| Valeria root | 100 | 110 | 99.5 | 100 | 110 |
| 5-HTP | 50 | 50 | 50 | 50 | 40 |
| Melatonin | 0.5 | 0.5 | 1 | 0.5 | 1 |
| Excipients** | 114.5 | 114.5 | 114.5 | 114.5 | 114.5 |

**microcrystalline cellulose, magnesium stearate, silicon dioxide.

TABLE XI

| | |
|---|---|
| Ingredients | Example No. |

| Amounts in mg | 51 | 52 | 53 | 54 | 55 |
|---|---|---|---|---|---|
| γ-aminobutyric acid | 100 | 100 | 130 | 115 | 105 |
| Magnolia bark | 100 | 110 | 100 | 90 | 110 |
| extract | | | | | |
| Valeria root | 100 | 110 | 90 | 100 | 110 |
| 5-HTP | 50 | 50 | 50 | 50 | 40 |
| Melatonin | 0.5 | 0.5 | 1 | 0.5 | 1 |
| Excipients** | 129.5 | 129.5 | 129.5 | 129.5 | 129.5 |

**microcrystalline cellulose, magnesium stearate, silicon dioxide.

TABLE XII

| | |
|---|---|
| Ingredients | Example No. |

| Amounts in mg | 56 | 57 | 58 | 59 | 60 |
|---|---|---|---|---|---|
| γ-aminobutyric acid | 100 | 105 | 100 | 110 | 100 |
| Magnolia bark | 100 | 110 | 100 | 90 | 110 |
| extract | | | | | |
| Valeria root | 100 | 110 | 90 | 100 | 110 |
| 5-HTP | 50 | 50 | 50 | 50 | 40 |
| Melatonin | 1 | 1 | 2 | 2 | 2 |
| Excipients** | 129.5 | 129.5 | 129.5 | 129.5 | 129.5 |

**microcrystalline cellulose, magnesium stearate, silicon dioxide.

Acceptable carriers or diluents are well known in the art, and are described, for example, in the incorporated material of Remington: The Science and Practice of Pharmacy (20.sup.th ed, Lippincott Williams & Wilkens Publishers (2003)), which is incorporated herein by reference in its entirety. The term “carrier” material or “excipient” herein can mean any substance, not itself a therapeutic agent, used as a carrier and/or diluent and/or adjuvant, or vehicle for delivery of a therapeutic agent to a subject or added to a composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants.

In one embodiment the excipients are microcrystalline cellulose, magnesium stearate, and silicon dioxide. Formulations for oral administration can be in dosages suitable for such administration.

One can also administer the disclosed compositions in sustained release forms or from sustained release drug delivery systems. A description of representative sustained release materials can be found in the incorporated materials in Remington: The Science and Practice of Pharmacy (20.sup.th ed, Lippincott Williams & Wilkens Publishers (2003)).

### Methods

Disclosed herein are methods for providing sustained natural sleep wherein the subject using the disclosed compositions has a restful, sustained sleep without drowsiness the next day. The disclosed methods comprise a subject self-administering or administering to a subject a composition, comprising:
A) from about 70% to about 90% by weight of active ingredients; and
B) from about 10% to about 30% by weight of carriers, excipients, adjunct ingredients, and formulation aides.

In one aspect the disclosed method for providing sustained natural sleep, comprises administering to a subject in need of sustained natural sleep a composition comprising:
A) from about 70% to about 90% by weight of active ingredients; and
B) from about 10% to about 30% by weight of carriers, excipients, adjunct ingredients, and formulation aides.

In one embodiment of this method the composition comprises:
A) from about 75% to about 85% by weight of an active ingredient component, comprising:
   a) from about 25% to about 38% by weight of aminophenylbutyric acid hydrochloride;
   b) from about 15% to about 28% by weight of magnolia bark;
   c) from about 24.5% to about 28% by weight of valerian root;
   d) from about 12% to about 14% by weight of 5-HTP; and
   e) from about 0.1 to about 1% by weight of melatonin; and
B) from about 15% to about 25% by weight carriers, excipients, and formulation aides.

In another embodiment of this method the composition comprises:
A) from about 75% to about 85% by weight of an active ingredient component, comprising:
   a) from about 35% to about 38% by weight of aminophenylbutyric acid hydrochloride;
   b) from about 24.5% to about 26% by weight of magnolia bark;
   c) from about 24.5% to about 26% by weight of valerian root;
   d) from about 12% to about 13% by weight of 5-HTP; and
   e) from about 0.1 to about 1% by weight of melatonin; and
B) from about 15% to about 25% by weight carriers, excipients, and formulation aides.

In another aspect the disclosed method for providing sustained natural sleep, comprises administering to a subject in need of sustained natural sleep a composition comprising:
A) from about 350 mg to about 450 mg of active ingredients; and
B) from about 80 mg to about 18 mg by weight of carriers, excipients, adjunct ingredients, and formulation aides.

In one embodiment of this method the composition comprises:
A) an active ingredient component, comprising:
   a) about 150 mg of aminophenylbutyric acid hydrochloride;
   b) about 100 mg of magnolia bark;
   c) about 100 mg of valerian root;
   d) about 50 mg of 5-HTP; and
   e) about 0.5 mg of melatonin; and
B) about 114.5 mg carriers, excipients, and formulation aides.

In another embodiment of this method the composition comprises:
A) an active ingredient component, comprising:
   a) about 150 mg of aminophenylbutyric acid hydrochloride;
   b) about 100 mg of magnolia bark;
   c) about 100 mg of valerian root;
   d) about 50 mg of 5-HTP; and
   e) about 0.5 mg of melatonin; and
B) about 98.5 mg carriers, excipients, and formulation aides.

In a further embodiment of this method the composition comprises:
A) an active ingredient component, comprising:
   a) about 150 mg of aminophenylbutyric acid hydrochloride;
   b) about 100 mg of magnolia bark;
   c) about 100 mg of valerian root;
   d) about 50 mg of 5-HTP; and
   e) about 0.5 mg of melatonin; and
B) about 129 mg carriers, excipients, and formulation aides.

In another embodiment of the disclosed method the composition comprises:
A) from about 75% to about 85% by weight of an active ingredient component, comprising:
   a) from about 25% to about 38% by weight of γ-aminobutyric acid;
   b) from about 15% to about 28% by weight of magnolia bark;
   c) from about 24.5% to about 28% by weight of valerian root;
   d) from about 12% to about 14% by weight of 5-HTP; and
   e) from about 0.1 to about 1% by weight of melatonin; and
B) from about 15% to about 25% by weight carriers, excipients, and formulation aides.

In another embodiment of this method the composition comprises:
A) from about 75% to about 85% by weight of an active ingredient component, comprising:
   a) from about 35% to about 38% by weight of γ-aminobutyric acid; b) from about 24.5% to about 26% by weight of magnolia bark;
   c) from about 24.5% to about 26% by weight of valerian root;
   d) from about 12% to about 13% by weight of 5-HTP; and
   e) from about 0.1 to about 1% by weight of melatonin; and
B) from about 15% to about 25% by weight carriers, excipients, and formulation aides.

In another aspect the disclosed method for providing sustained natural sleep, comprises administering to a subject in need of sustained natural sleep a composition comprising:
A) from about 350 mg to about 450 mg of active ingredients; and
B) from about 80 mg to about 18 mg by weight of carriers, excipients, adjunct ingredients, and formulation aides.

In one embodiment of this method the composition comprises:
A) an active ingredient component, comprising:
   a) about 100 mg of γ-aminobutyric acid;
   b) about 100 mg of magnolia bark;
   c) about 100 mg of valerian root;
   d) about 50 mg of 5-HTP; and
   e) about 0.5 mg of melatonin; and
B) about 114.5 mg carriers, excipients, and formulation aides.

In another embodiment of this method the composition comprises:
A) an active ingredient component, comprising:
   a) about 100 mg of γ-aminobutyric acid;
   b) about 100 mg of magnolia bark;
   c) about 100 mg of valerian root;
   d) about 50 mg of 5-HTP; and
   e) about 0.5 mg of melatonin; and
B) about 98.5 mg carriers, excipients, and formulation aides.

In a further embodiment of this method the composition comprises:
A) an active ingredient component, comprising:
   a) about 100 mg of γ-aminobutyric acid;
   b) about 100 mg of magnolia bark;
   c) about 100 mg of valerian root;
   d) about 50 mg of 5-HTP; and
   e) about 0.5 mg of melatonin; and
B) about 129 mg carriers, excipients, and formulation aides.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A composition comprising:
A) from about 70% to about 90% by weight of active ingredients; and
B) from about 10% to about 30% by weight of carriers, excipients, adjunct ingredients, and formulation aides.

2. The composition according to claim 1, comprising:
A) from about 75% to about 85% by weight of an active ingredient component, comprising:
a) from about 25% to about 38% by weight of γ-aminobutyric acid;
b) from about 15% to about 28% by weight of magnolia bark;
c) from about 24.5% to about 28% by weight of valerian root;
d) from about 12% to about 14% by weight of 5-HTP; and
e) from about 0.1 to about 1% by weight of melatonin; and
B) from about 15% to about 25% by weight carriers, excipients, and formulation aides.

3. The composition according to claim 1, comprising:
A) from about 75% to about 85% by weight of an active ingredient component, comprising:
a) from about 35% to about 38% by weight of γ-aminobutyric acid;
b) from about 24.5% to about 26% by weight of magnolia bark;
c) from about 24.5% to about 26% by weight of valerian root;
d) from about 12% to about 13% by weight of 5-HTP; and
e) from about 0.1 to about 1% by weight of melatonin; and
B) from about 15% to about 25% by weight carriers, excipients, and formulation aides.

4. A composition, comprising:
A) from about 350 mg to about 450 mg of active ingredients; and
B) from about 80 mg to about 18 mg by weight of carriers, excipients, adjunct ingredients, and formulation aides.

5. The composition according to claim 4, comprising:
A) an active ingredient component, comprising:
a) about 100 mg of γ-aminobutyric acid;
b) about 100 mg of magnolia bark;
c) about 100 mg of valerian root;
d) about 50 mg of 5-HTP; and
e) about 0.5 mg of melatonin; and
B) about 114.5 mg carriers, excipients, and formulation aides.

6. The composition according to claim 4, comprising:
A) an active ingredient component, comprising:
a) about 100 mg of γ-aminobutyric acid;
b) about 100 mg of magnolia bark;
c) about 100 mg of valerian root;
d) about 50 mg of 5-HTP; and
e) about 0.5 mg of melatonin; and
B) about 98.5 mg carriers, excipients, and formulation aides.

7. The composition according to claim 4, comprising:
A) an active ingredient component, comprising:
a) about 100 mg of γ-aminobutyric acid;
b) about 100 mg of magnolia bark;
c) about 100 mg of valerian root;
d) about 50 mg of 5-HTP; and
e) about 0.5 mg of melatonin; and
B) about 129 mg carriers, excipients, and formulation aides.

8. A method for providing sustained natural sleep, comprising administering to a subject in need of sustained natural sleep a composition comprising:
A) from about 70% to about 90% by weight of active ingredients; and
B) from about 10% to about 30% by weight of carriers, excipients, adjunct ingredients, and formulation aides.

9. The method according to claim 8, wherein the composition comprises:
A) from about 75% to about 85% by weight of an active ingredient component, comprising:
a) from about 25% to about 38% by weight of γ-aminobutyric acid;
b) from about 15% to about 28% by weight of magnolia bark;
c) from about 24.5% to about 28% by weight of valerian root;
d) from about 12% to about 14% by weight of 5-HTP; and
e) from about 0.1 to about 1% by weight of melatonin; and
B) from about 15% to about 25% by weight carriers, excipients, and formulation aides.

10. The method according to claim 8, wherein the composition comprises:
A) from about 75% to about 85% by weight of an active ingredient component, comprising:
a) from about 35% to about 38% by weight of γ-aminobutyric acid;
b) from about 24.5% to about 26% by weight of magnolia bark;
c) from about 24.5% to about 26% by weight of valerian root;
d) from about 12% to about 13% by weight of 5-HTP; and
e) from about 0.1 to about 1% by weight of melatonin; and
B) from about 15% to about 25% by weight carriers, excipients, and formulation aides.

11. A method for providing sustained natural sleep, comprising administering to a subject in need of sustained natural sleep a composition comprising:
A) from about 350 mg to about 450 mg of active ingredients; and
B) from about 80 mg to about 18 mg by weight of carriers, excipients, adjunct ingredients, and formulation aides.

12. The method according to claim 11, wherein the composition comprises:
A) an active ingredient component, comprising:
a) about 100 mg of γ-aminobutyric acid;
b) about 100 mg of magnolia bark;
c) about 100 mg of valerian root;
d) about 50 mg of 5-HTP; and
e) about 0.5 mg of melatonin; and
B) about 114.5 mg carriers, excipients, and formulation aides.

13. The method according to claim 11, wherein the composition comprises:
A) an active ingredient component, comprising:
a) about 100 mg of γ-aminobutyric acid;
b) about 100 mg of magnolia bark;
c) about 100 mg of valerian root;
d) about 50 mg of 5-HTP; and
e) about 0.5 mg of melatonin; and
B) about 98.5 mg carriers, excipients, and formulation aides.

14. The method according to claim 11, wherein the composition comprises:
A) an active ingredient component, comprising:
a) about 100 mg of γ-aminobutyric acid;
b) about 100 mg of magnolia bark;
c) about 100 mg of valerian root;
d) about 50 mg of 5-HTP; and
e) about 0.5 mg of melatonin; and
B) about 129 mg carriers, excipients, and formulation aides.
